(19) United States
(12) Patent Application Publication
(10) Pub. No.: US 2015/0212074 A1
(43) Pub. Date: Jul. 30, 2015

(54) IMMUNOASSAY RAPID DIAGNOSTIC TEST UNIVERSAL ANALYSIS DEVICE, SYSTEM, METHOD AND COMPUTER READABLE MEDIUM

(71) Applicant: FIO CORPORATION, Toronto (CA)

(72) Inventors: Qing Xiang, Toronto (CA); Jeongjin Lee, Vaughan (CA)

(21) Appl. No.: 14/416,903

(22) PCT Filed: Jul. 24, 2013
(86) PCT No.: PCT/CA2013/000670

§ 371 Date: Jan. 23, 2015

Related U.S. Application Data
(60) Provisional application No. 61/675,049, filed on Jul. 24, 2012.

(51) Int. Cl. G01N 33/53; G01N 33/543; G01N 21/64

(52) U.S. Cl. CPC G01N 33/5302; G01N 21/6456; G01N 33/54366; G01N 2201/062

(57) ABSTRACT

A device, system, method, and computer readable medium is provided for universal analysis of immunoassay rapid diagnostic tests. They permit different tests, from varied manufacturers, to be read even though such tests may be associated with reflection and/or emission types of signals. The device, system, method, and computer readable medium use one or more rapid diagnostic test databases, including information on products commercially available on the market and on customized rapid tests. The device, system, method, and computer readable medium identify the type of test to be analyzed by matching it with the test database(s). They capture a corresponding reflection and/or emission signal from the test, as appropriate. The device, system, method, and computer readable medium may transform the signal to an image, or vice-versa, and/or analyze the image to interpret the test result.

### FIELD OF THE INVENTION

The present invention relates generally to an rapid diagnostic test analysis device and method, and more particularly to an immunoassay rapid diagnostic test universal analysis device, system, method and computer readable medium.

### BACKGROUND OF THE INVENTION

In the prior art, the rapid immunoassay diagnostic test may have achieved great commercial application in many fields—potentially including, for example, in association with pregnancy tests, infectious disease tests, oncology tests, cardiovascular disease tests, animal health tests, and/or tests related to foods and the environment, etc. One of the most successful examples may be all kinds of lateral flow immunoassay tests.

Usually, lateral flow types of rapid tests may have been visually interpreted by end users. Such visual interpretation by end users may have been subject to inconsistency and/or human interference. There may be a trend to using devices to analyze the results of such tests. There may be a number of US patents and/or published US patent applications which may have disclosed methods and/or devices to automatically analyze lateral flow tests. There may be a great amount of diversity in the various prior art lateral flow cassettes, whether by application, manufacture, and/or signal format. It may have been almost impossible for a device to read more than one type of these products. Typically, prior art devices for reading lateral flow tests may have been adapted to read just one specific lateral flow product and/or test.

Even were it not so, prior art devices may have been adapted to just read only a single type of test—i.e., either reflection signals (e.g., from a colloidal gold and/or colored latex bead type of test) or emission signal (e.g., from a fluorescence type of test).

Furthermore, prior art devices manufactured by any particular company may have been adapted just to read test products which were also manufactured by that particular company. In order to read a different product, such devices would have needed to be customized and/or reconfigured based on differences in physical appearance, detection areas, control lines and test line positions in the various products. Such an endeavor might have been highly unlikely to have been performed, if at all.

The present invention may preferably, but need not necessarily, provide a method, system and/or device to universally analyze various immunoassay rapid diagnostic tests.

The present invention may preferably address, mitigate, alleviate, and/or overcome one or more of the aforementioned disadvantages, shortcomings, problems and/or other issues associated with the prior art, and/or to achieve one or more of the aforementioned objects of the invention.

### SUMMARY OF THE INVENTION

According to the invention, there is disclosed a device for analysis of various test cassettes for immunoassay rapid diagnostic tests. The device includes one or more walls defining an enclosed chamber, which is adapted to selectively receive the various cassettes one at a time, each as a loaded cassette, in selectively removable relation. The device also includes two or more light emitting diodes (LEDs) within the chamber, which include at least one reflection light emitting diode (LED) adapted to illuminate the loaded cassette when lit, and at least one fluorescent LED adapted to fluoresce the loaded cassette when lit. The device also includes an imaging element inside the chamber, which automatically captures a first image of the loaded cassette when at least one of the LEDs is lit. The device also includes at least one processor which, with reference to the first image and to a database that comprises at least one test protocol associated with each of the various test cassettes, automatically identifies an applicable test protocol for the loaded cassette. The processor automatically, after incubation of the loaded cassette and/or depending on the applicable test protocol: (i) determines when the first image captures a post-incubation test signal from the loaded cartridge; and/or otherwise (ii) lights the fluorescent LED to generate the test signal by emission, and/or the reflection LED to generate the test signal by reflection, from the loaded cassette and uses the imaging element to capture a second image of the loaded cassette and of the test signal. The processor automatically provides the test signal, in the first image and/or the second image, for analysis of the loaded cassette.

According to an aspect of one preferred embodiment of the invention, the processor may preferably, but need not necessarily, automatically analyze the test signal, preferably with reference to the applicable test protocol for the loaded cassette.

According to an aspect of one preferred embodiment of the invention, the test signal may preferably, but need not necessarily, include a test line signal, preferably corresponding to a test line, preferably present on the loaded cassette after incubation. The processor may preferably, but need not necessarily, automatically measure an intensity of the test line signal.

According to an aspect of one preferred embodiment of the invention, the test signal may preferably, but need not necessarily, include a control line signal, preferably corresponding to a control line, preferably present on the loaded cassette after incubation. The processor may preferably, but need not necessarily, automatically measure an intensity of the control line signal.

According to an aspect of one preferred embodiment of the invention, the applicable test protocol may preferably, but need not necessarily, include a predetermined assay threshold value. The processor may preferably, but need not necessarily, analyze the test signal, preferably with reference to the assay threshold value and/or to automatically determine a diagnostic test result associated with the loaded cassette.

According to an aspect of one preferred embodiment of the invention, the device may preferably, but need not necessarily, include an output device which may preferably, but need not necessarily, automatically present the diagnostic test result to a user of the device.

According to an aspect of one preferred embodiment of the invention, the device may preferably, but need not necessarily, include a memory onboard the device which may preferably, but need not necessarily, store one or more sets of executable instructions, preferably to encode the processor and/or to automatically analyze the test signal as aforesaid.

According to an aspect of one preferred embodiment of the invention, the device may preferably, but need not necessarily, include a memory onboard the device which may preferably, but need not necessarily, store one or more sets of executable instructions, preferably to encode the processor and/or to automatically identify the applicable test protocol as aforesaid.

According to an aspect of one preferred embodiment of the invention, the device may preferably, but need not necessarily, include a memory onboard the device which may preferably, but need not necessarily, store the database.

According to an aspect of one preferred embodiment of the invention, the device may preferably, but need not necessarily, include a communications element onboard the device which the processor may preferably, but need not necessarily, automatically use to remotely reference the database, preferably as aforesaid.

According to an aspect of one preferred embodiment of the invention, the device may preferably, but need not necessarily, include at least one optical filter. The processor may preferably, but need not necessarily, automatically ensure the optical filter is moved to an engaged position, preferably between the loaded cassette and the imaging element and/or preferably before lighting a corresponding emission LED. The imaging element may preferably, but need not necessarily, then capture the test signal through the optical filter. The processor may preferably, but need not necessarily, automatically ensure the optical filter is moved to a disengaged position, preferably clear of the imaging element and/or preferably before lighting the reflection LED. The imaging element may preferably, but need not necessarily, then capture the test signal clear of the optical filter.

According to an aspect of one preferred embodiment of the invention, the optical filter may preferably, but need not necessarily, be mounted on a sliding switch which may preferably, but need not necessarily, slide the optical filter, preferably between the engaged position and the disengaged position.

According to an aspect of one preferred embodiment of the invention, the optical filter may preferably, but need not necessarily, be mounted on a rotatable mechanism which may preferably, but need not necessarily, rotate the optical filter, preferably between the engaged position and the disengaged position.

According to an aspect of one preferred embodiment of the invention, the optical filter may preferably, but need not necessarily, be an optical band pass filter.

According to an aspect of one preferred embodiment of the invention, the device may preferably, but need not necessarily, include an optical long pass filter, preferably positioned between the loaded cassette and the imaging element. The imaging element may preferably, but need not necessarily, capture the test signal through the optical long pass filter.

According to an aspect of one preferred embodiment of the invention, the aforesaid at least one reflection LED may preferably, but need not necessarily, include at least one white LED.

According to an aspect of one preferred embodiment of the invention, the aforesaid at least one fluorescent LED may preferably, but need not necessarily, include at least one ultraviolet LED.

According to an aspect of one preferred embodiment of the invention, the aforesaid at least one fluorescent LED may preferably, but need not necessarily, include at least one colored LED.

According to an aspect of one preferred embodiment of the invention, the processor may preferably, but need not necessarily, identify the applicable test protocol, preferably with reference to one or more of the following which may preferably, but need not necessarily, be captured in the first image and/or the second image, and/or stored in the database for the loaded cassette: one or more cassette dimensions; one or more cassette shapes; one or more detection line dimensions; one or more control line dimensions; one or more detection areas; one or more membrane areas; one or more control line positions; one or more test line positions; one or more cassette colors; one or more line colors; manufacturer indicia; product indicia; brand name indicia; application indicia; disease indicia; test type indicia; incubation time indicia; expected results indicia; barcodes; two-dimensional barcodes; labels; and/or other printed and written indicia.

According to an aspect of one preferred embodiment of the invention, the processor may preferably, but need not necessarily, additionally identify the applicable test protocol, preferably with reference to one or more of the following which may preferably, but need not necessarily, be received by the device from the loaded cassette in the first image, in the second image and/or otherwise, and/or stored in the database for the loaded cassette: magnetically stored data, fluorescence data, and/or radioactive signal data.

According to an aspect of one preferred embodiment of the invention, the device may preferably, but need not necessarily, be adapted for analysis of various lateral flow cassettes, preferably as the test cassettes.

According to an aspect of one preferred embodiment of the invention, the device may preferably, but need not necessarily, be adapted for use with a cellular telephone which may preferably, but need not necessarily, provide at least one of the imaging element and the processor.

According to an aspect of one preferred embodiment of the invention, left and/or right channel audio signals, preferably from the cellular telephone, may preferably, but need not necessarily, be adapted to turn the reflection LED and/or the fluorescent LED on and/or off.

According to an aspect of one preferred embodiment of the invention, the imaging element may preferably, but need not necessarily, include one or more scanning heads.

According to the invention, there is also disclosed a system for analysis of various test cassettes for immunoassay rapid diagnostic tests. The system includes one or more walls defining an enclosed chamber, which is adapted to selectively receive the various cassettes one at a time, each as a loaded cassette, in selectively removable relation. The system also includes two or more light emitting diodes (LEDs) within the chamber, which include at least one reflection light emitting diode (LED) adapted to illuminate the loaded cassette when lit, and at least one fluorescent LED adapted to fluoresce the loaded cassette when lit. The system also includes a database, which includes at least one test protocol associated with each of the various test cassettes. The system also includes a cellular telephone received by the walls of the chamber. The cellular telephone includes: a camera inside the chamber which automatically captures a first image of the loaded cassette when at least one of the LEDs is lit; and at least one processor which, with reference to the first image and in communication with the database, automatically identifies an applicable test protocol for the loaded cassette. The processor automatically, after incubation of the loaded cassette and/or depending on the applicable test protocol: (i) determines when the first image captures a post-incubation test signal from the loaded cartridge; and/or otherwise (ii) lights the fluorescent LED to generate the test signal by emission, and/or the reflection LED to generate the test signal by reflection, from the loaded cassette and uses the imaging element to capture a second image of the loaded cassette and of the test signal. The processor automatically provides the test signal, in the first image and/or the second image, for analysis of the loaded cassette.

According to an aspect of one preferred embodiment of the invention, left and/or right channel audio signals, preferably from the cellular telephone, may preferably, but need not necessarily, be adapted to turn the reflection LED and/or the fluorescent LED on and/or off.

According to the invention, there is also disclosed a method for analysis of various test cassettes for immunoassay rapid diagnostic tests. The method includes a receiving step of selectively receiving the various cassettes one at a time, each as a loaded cassette, in selectively removable relation within an enclosed chamber which is defined by one or more walls. The method also includes a light emitting diode (LED) step of providing two or more LEDs within the chamber, which include at least one reflection LED adapted to illuminate the loaded cassette when lit, and at least one fluorescent LED adapted to fluoresce the loaded cassette when lit. The method also includes an imaging step of automatically capturing a first image of the loaded cassette, when at least one of the LEDs is lit, using an imaging element inside the chamber. The method also includes a database step of providing a database, which includes at least one test protocol associated with each of the various test cassettes. The method also includes a processing step of using at least one processor, with reference to the first image and to the database, automatically identifying an applicable test protocol for the loaded cassette. In the processing step, the processor automatically, after incubation of the loaded cassette and/or depending on the applicable one said test protocol: (i) determines when the first image captures a post-incubation test signal from the loaded cartridge; and/or otherwise (ii) lights the fluorescent LED to generate the test signal by emission, and/or the reflection LED to generate the test signal by reflection, from the loaded cassette and uses the imaging element to capture a second image of the loaded cassette and of the test signal. In the processing step, the processor automatically provides the test signal, in the first image and/or the second image, for analysis of the loaded cassette.

According to an aspect of one preferred embodiment of the invention, preferably in the processing step, the processor may preferably, but need not necessarily, automatically analyze the test signal, preferably with reference to the applicable test protocol for the loaded cassette.

According to an aspect of one preferred embodiment of the invention, preferably in the processing step, the test signal may preferably, but need not necessarily, include a test line signal, preferably corresponding to a test line, preferably present on the loaded cassette after incubation. The processor may preferably, but need not necessarily, automatically measure an intensity of the test line signal.

According to an aspect of one preferred embodiment of the invention, in the processing step, the test signal comprises a control line signal a test line signal, preferably corresponding to a control line, preferably present on the loaded cassette after incubation. The processor may preferably, but need not necessarily, automatically measure an intensity of the control line signal.

According to an aspect of one preferred embodiment of the invention, in the processing step, the applicable test protocol may preferably, but need not necessarily, include a predetermined assay threshold value. The processor may preferably, but need not necessarily, analyze the test signal, preferably with reference to the assay threshold value and/or preferably to automatically determine a diagnostic test result and/or preferably associated with the loaded cassette.

According to an aspect of one preferred embodiment of the invention, the method may preferably, but need not necessarily, include a presentation step, wherein the diagnostic test result may preferably, but need not necessarily, be automatically presented, preferably using an output device.

According to an aspect of one preferred embodiment of the invention, preferably before the processing step, one or more sets of executable instructions may preferably, but need not necessarily, be stored in a memory. Preferably in the processing step, the executable instructions may preferably, but need not necessarily, encode the processor, preferably to automatically analyze the test signal as aforesaid.

According to an aspect of one preferred embodiment of the invention, preferably before the processing step, one or more sets of executable instructions may preferably, but need not necessarily, be stored in a memory. Preferably in the processing step, the executable instructions may preferably, but need not necessarily, encode the processor, preferably to automatically identify the applicable test protocol as aforesaid.

According to an aspect of one preferred embodiment of the invention, preferably in the processing step, the processor may preferably, but need not necessarily, automatically use a communications element, preferably to remotely reference the database, preferably as aforesaid.

According to an aspect of one preferred embodiment of the invention, the method may preferably, but need not necessarily, also include a filtering step of providing at least one optical filter. Preferably in the processing step, (i) the processor may preferably automatically ensure the optical filter may preferably, but need not necessarily, be moved to an engaged position, preferably between the loaded cassette and the imaging element and/or preferably before lighting a corresponding emission LED. The imaging element may preferably, but need not necessarily, then capture the test signal through the optical filter. And/or, preferably in the processing step, (ii) the processor may preferably automatically ensure the optical filter may preferably, but need not necessarily, be moved to a disengaged position, preferably clear of the imaging element and/or preferably before lighting the reflection LED. The imaging element may preferably, but need not necessarily, then capture the test signal clear of the optical filter.

According to an aspect of one preferred embodiment of the invention, the method may preferably, but need not necessarily, also include a filtering step of providing an optical long pass filter, preferably positioned between the loaded cassette and the imaging element. In the processing step, the imaging element may preferably, but need not necessarily, capture the test signal, preferably through the optical long pass filter.

According to an aspect of one preferred embodiment of the invention, preferably in the LED step, at least one white LED may preferably, but need not necessarily, be provided as the aforesaid at least one reflection LED.

According to an aspect of one preferred embodiment of the invention, preferably in the LED step, at least one ultraviolet LED may preferably, but need not necessarily, be provided as the aforesaid at least one fluorescent LED.

According to an aspect of one preferred embodiment of the invention, preferably in the LED step, at least one colored LED may preferably, but need not necessarily, be provided as the aforesaid at least one fluorescent LED.

According to an aspect of one preferred embodiment of the invention, preferably in the processing step, the processor may preferably, but need not necessarily, identify the applicable test protocol with reference to one or more of the following which may preferably, but need not necessarily, be captured in the first image and/or the second image, and/or stored in the database for the loaded cassette: one or more cassette dimensions; one or more cassette shapes; one or more detection line dimensions; one or more control line dimensions; one or more detection areas; one or more membrane areas; one or more control line positions; one or more test line positions; one or more cassette colors; one or more line colors; manufacturer indicia; product indicia; brand name indicia; application indicia; disease indicia; test type indicia; incubation time indicia; expected results indicia; barcodes; two-dimensional barcodes; labels; and/or other printed and written indicia.

According to an aspect of one preferred embodiment of the invention, preferably in the processing step, the processor may preferably, but need not necessarily, additionally identify the applicable test protocol with reference to one or more of the following which may preferably, but need not necessarily, be received from the loaded cassette in the first image, in the second image and/or otherwise, and/or stored in the database for the loaded cassette: magnetically stored data; fluorescence data; and/or radioactive signal data.

According to an aspect of one preferred embodiment of the invention, preferably at least in the imaging step and/or the processing step, a cellular telephone may preferably, but need not necessarily, be provided as the aforesaid imaging element and/or as the aforesaid processor.

According to an aspect of one preferred embodiment of the invention, preferably at least in the imaging step and/or the processing step, left and/or right channel audio signals, preferably from the cellular telephone, may preferably, but need not necessarily, turn the reflection LED and/or the fluorescent LED on and/or off.

According to an aspect of one preferred embodiment of the invention, preferably in the imaging step, one or more scanning heads may preferably, but need not necessarily, be provided, preferably as at least part of the aforesaid imaging element.

According to the invention, there is also disclosed a computer readable medium for analysis of various test cassettes for immunoassay rapid diagnostic tests. The computer readable medium is for use with one or more walls defining an enclosed chamber, which is adapted to selectively receive the various cassettes one at a time, each as a loaded cassette, in selectively removable relation. The computer readable medium is also for use with two or more light emitting diodes (LEDs) within the chamber, which include at least one reflection light emitting diode (LED) adapted to illuminate the loaded cassette when lit, and at least one fluorescent LED adapted to fluoresce the loaded cassette when lit. The computer readable medium is also for use with an imaging element inside the chamber. The computer readable medium is also for use with a database which includes at least one test protocol associated with each of the various test cassettes. The computer readable medium includes executable instructions, which are physically stored thereon. The instructions, upon execution, encode at least one processor to automatically capture a first image of the loaded cassette using the imaging element when at least one of the LEDs is lit. The instructions, upon execution, encode the aforesaid at least one processor to automatically identify, with reference to the first image and to the database, an applicable test protocol for the loaded cassette. The instructions, upon execution, encode the aforesaid at least one processor to automatically, after incubation of the loaded cassette and/or depending on the applicable one said test protocol: (i) determine when the first image captures a post-incubation test signal from the loaded cartridge; and/or otherwise (ii) light the fluorescent LED to generate the test signal by emission, and/or the reflection LED to generate the test signal by reflection, from the loaded cassette and uses the imaging element to capture a second image of the loaded cassette and of the test signal. The instructions, upon execution, encode the aforesaid at least one processor to automatically provide the test signal, in the first image and/or the second image, for analysis of the loaded cassette.

According to the invention, there is also disclosed a device, system and/or method which may be configured for use in association with one or more lateral flow product databases.

These databases, and/or any others provided and/or called for according to the invention, may preferably, but need not necessarily, be local to the device, to other components of the system, and/or to the computer readable medium, and/or they may preferably, but need not necessarily, be remote therefrom, preferably with a communications element being used to remotely access and/or reference them. According to the invention, the databases may may preferably, but need not necessarily, take the form of one or more local, remote, distributed, congruent and/or peer-to-peer databases, which may preferably, but need not necessarily, be accessible by the device locally and/or over one or more of its regular wireless (and/or wired) communication networks, including terrestrial and/or satellite networks—e.g., the Internet and/or cloud-based networks.

Preferably, the databases may comprise, for each lateral flow product, a collection of data about manufacturer information, product information, test type, identification information, incubation time, and/or expected results, etc. Manufacturer information stored in the databases may preferably comprise information concerning the manufacturer of the product. Product information stored in the databases may preferably comprise information concerning product brand names and/or application information. Identification data stored in the databases may preferably comprise specific data which can be used to discriminate the individual product from others, and/or which can be used to identify the test by the device, system and/or method according to the present invention. Specific data may include data concerning one or more physical dimensions and/or colors associated with the product, and/or it may include and/or be encoded in writing and/or prior art labeling techniques (and/or other indicia, labels, writing, printing, markings, text, characters and/or symbols, with these terms understood by persons skilled in the art to be capable of being used interchangeably mutatis mutandis herein) which may be associated with a particular product and/or test, such as, for example, barcode images, magnetically stored data, fluorescence data, and/or radioactive signal data, etc.

According to one aspect of the present invention, test identification may preferably be performed in a process by which the device, system and/or method may preferably automatically recognize a cassette type and/or its manufacturer. Preferably, a pre-defined assay cut-off value (e.g., a positive and/or negative threshold) may be applied for that type of cassette for diagnostic purposes.

According to one aspect of the present invention, the device, system and/or method may preferably, but need not necessarily, include an electronic board, a processor (e.g., a microprocessor), image analysis and/or processing software, a light chamber, one or more white (cassette recognition, reflection) LEDs, one or more color (emission) LEDs and/or ultraviolet (“UV”) LEDs, an optical filter, and/or a color camera. Preferably, the optical filter may be an optical band pass filter and/or an optical long pass filter. Preferably, the optical filter may be mounted on a sliding switch and/or on a rotatable mechanism, and/or it may be moved towards and/or away from the front of the camera. Preferably, the LEDs may be illuminated (i.e., on) to permit the camera to take a picture, and may preferably just illuminate the light chamber when the camera is taking a picture. The processor and/or controlling electronics may control whether one or more of the LEDs is on or off. The device, system and/or method may preferably enable one or more lateral flow cassettes to be received for recognition and analysis. The microprocessor may preferably be adapted with image processing software for analysis of one or more images of the lateral flow cassettes.

According to one aspect of the present invention, for reading reflection signal type of tests (e.g., colloidal gold and/or colored latex bead types of tests), the white LEDs may preferably be on. [References herein to use of the invention with colloidal gold tests and/or colored latex bead tests may be considered, more generally, as references to its use (if and when appropriate) with reflection signal tests, with any changes which may be necessary and/or appropriate for such use.] In such circumstances, the color and/or UV LEDs may preferably be off. The optical filter may preferably be switched away from the color camera. Preferably, the color camera may take an image of the entire cassette, preferably including the plastic case and/or membrane area.

According to one aspect of the present invention, the image taken by the camera may preferably be analyzed by the image analysis and/or processing software in the microprocessor. [Each reference herein to image analysis software and/or to image processing software may be considered as a reference (if and when appropriate) to the other, to imaging software, and/or to software more generally, with any changes which may be necessary and/or appropriate in such instance.] The function of the image analysis software may preferably include: (1) cassette recognition, preferably with reference to cassette features (such as for example size, aspect ratio, color, shape, letters, etc.) and/or so as to provide a user with information concerning the cassette manufacturer, disease to be tested, etc.; (2) membrane region identification; (3) measurement of the intensity of the test line and/or control line in the membrane region and/or area; and/or (4) presenting diagnostic results according to one or more cut-off values, preferably as pre-set in the software.

The image processing software may preferably assess one or more images to determine and/or present the user cassette type and the diagnostic results to a user of the device, system and/or method according to the invention.

According to one aspect of the present invention, for reading emission signal type of tests (e.g., fluorescent lateral flow cassette tests), two or more images may preferably but need not necessarily be taken for each test. [References herein to use of the invention with fluorescent signal tests may be considered, more generally, as references to its use (if and when appropriate) with emission signal tests, with any changes which may be necessary and/or appropriate for such use.] In such circumstances, the first image may preferably be an image of the cassette as taken with the white LED on, and the color LEDs off, and with the optical filter (e.g., an optical band pass filter) moved away from the camera. [Each reference herein to an optical band pass filter and/or to an optical long pass filter may be considered as a reference (if and when appropriate) to optical filters, more generally, and/or to another specific type of optical filter, with any changes which may be necessary and/or appropriate for such filter.] Preferably, after the first image, a further image (e.g., the second image) may be a fluorescent signal image of the membrane region, preferably with the color LEDs on and/or the white LEDs off, and preferably with the optical filter (e.g., the optical band pass filter) in front of the camera. Preferably, for emission signal tests, there may be no line visible in the membrane area in the first image because, preferably, the color and/or UV LEDs (which may be required for fluorescent lateral flow images) may be off. [Each reference herein to color LEDs and/or to UV LEDs may be considered as a reference (if and when appropriate) to the other and/or to non-white LEDs, more generally, with any changes which may be necessary and/or appropriate for such LEDs.] The first image may preferably enable software according to the invention to perform cassette recognition. Preferably, the fluorescent control line and/or test signal line may be present and/or visible in the second image. [According to one aspect of the present invention, the fluorescent control line and/or test signal line may be rendered visible by the optical filter.] The image processing software may preferably combine and/or assess the two or more images to determine and/or present the user cassette type and the diagnostic results to a user of the device, system and/or method according to the invention.

Other advantages, features and characteristics of the present invention, as well as methods of operation and functions of the related elements of the device, system, method, and computer readable medium and the combination of steps, parts and economies of manufacture, will become more apparent upon consideration of the following detailed description and the appended claims with reference to the accompanying drawings, the latter of which are briefly described hereinbelow.

### BRIEF DESCRIPTION OF THE DRAWINGS

The novel features which are believed to be characteristic of the device, system, method, and computer readable medium according to the present invention, as to the structure, organization, use, and method of operation, together with further objectives and advantages thereof, will be better understood from the following drawings in which presently preferred embodiments of the invention will now be illustrated by way of example. It is expressly understood, however, that the drawings are for the purpose of illustration and description only, and are not intended as a definition of the limits of the invention. In the accompanying drawings:

FIG. 1 is a schematic diagram of a universal lateral flow reader device according to a preferred embodiment of the invention;

FIG. 2 depicts three images of three different prior art cassettes made by different manufacturers, taken according to a preferred embodiment of the invention;

FIG. 3 depicts two different images of a single fluorescent lateral flow cassette: (A) is a first image taken with white LEDs on, color LEDs off, and an optical filter away from a camera; and (B) is a second image showing a test line, taken with the white LEDs off, the color LEDs on, and the optical filter in front of the camera; both taken according to a preferred embodiment of the invention;

FIG. 4 is an image of a universal lateral flow reader system, including a cellular telephone, according to another preferred embodiment of the invention;

FIG. 5 depicts two images of a fluorescent lateral flow cassette: (A) is a first image taken with a white LED on, and an ultraviolet (UV) LED off; and (B) is a second image taken with the white LED off, and the UV LED on; both taken with a long pass optical filter in front of the camera according to a further preferred embodiment of the invention; and

FIG. 6 depicts four flowcharts, each showing a workflow method for lighting and incubation: (A) and (B) are flowcharts with a cassette incubated outside of a reader device; and (C) and (D) are flowcharts with the cassette incubated inside of the reader device; all according to different preferred embodiments of the invention.

### DETAILED DESCRIPTION OF PREFERRED EMBODIMENTS

Preferred embodiments of the device, system, method, and computer readable medium according to the invention are alternately herein referred to, collectively and/or individually, as the universal lateral flow reader, device, system, method and/or computer readable medium (or simply as the reader, device system, method and/or computer readable medium). References to one or more of the reader, device, system, method and/or computer readable medium may, if and as appropriate, be understood by persons having ordinary skill in the art to apply, mutatis mutandis, to the others.

Persons skilled in the art will appreciate that although some of the components, relations, functionalities and applications of the reader, device, system, method and computer readable medium are not specifically referenced or described in conjunction with each other, they may be used or adapted for use in association therewith. The reader, device, system, method and computer readable medium described herein are suitable for use with each other, but they are not so limited.

How It Works

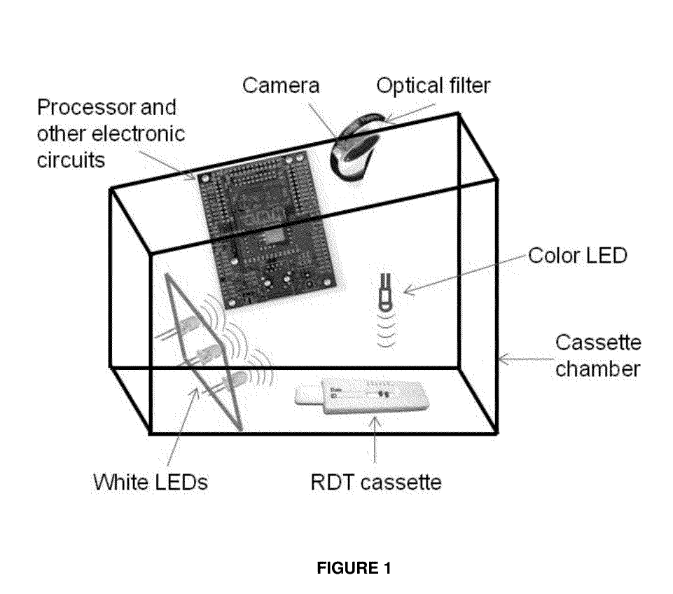

FIG. 1 provides a schematic diagram of one reader device and/or system (and/or for use with a method) according to a preferred embodiment of the invention. It preferably includes a color charge-coupled device (“CCD”) camera, white LEDs, color LEDs, an optical filter, a printed circuit board (“PCB”), and a lateral flow test cassette. The cassette is preferably enclosed in a chamber when a picture is taken. The white LEDs may have a wide illumination angle. Preferably, when on, the white LEDs may make the whole cassette chamber bright and provide a constant lighting environment for the camera. The color LEDs (and/or UV LEDs) are preferably used as an excitation source of one or more fluorophores in a fluorescence lateral flow test. The light of color LEDs may be collimated. The color LED light may illuminate only the membrane area of the cassette. The camera is preferably mounted high enough to take an image of the entire cassette. According to one preferred embodiment, the optical filter is an optical band pass filter which passes the fluorescent wavelength of the fluorophore.

Preferably, for gold lateral flow cassettes (and/or reflection signal tests), the white LEDs are on, the color LEDs are off, and the optical filter is switched away from the camera. The camera preferably takes an image of the entire cassette.

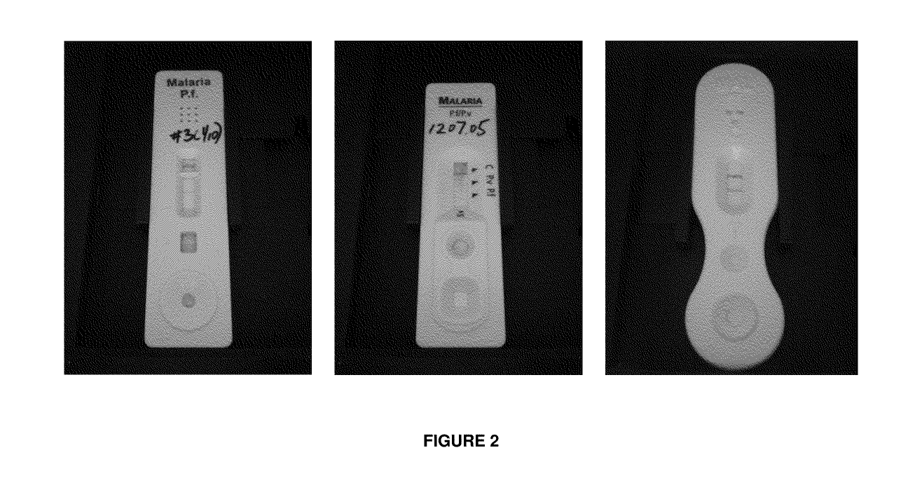

FIG. 2 shows images of cassettes from three different manufacturers, as may be taken according to a preferred embodiment of the invention. Those bright color images, as preferably illuminated by the white LEDs, are analyzed by software in the microprocessor. Preferably, some of the functions of the image analysis software include: (1) cassette recognition which may be performed with reference to cassette features such as, for example, cassette size, aspect ratio, color, shape, letters, etc.; (2) membrane region identification for each type of cassette, i.e., in a preferred embodiment according to the invention, identifying a precise and/or approximate location of a membrane region for each cassette as (and cassette type which may be) inserted into the chamber; (3) measurement of the intensity of one or more test lines and/or control lines in the membrane region; and (4) determination of diagnostic results. Preferably, for each type of cassette, an assay cut-off value may be tested and/or predetermined, and pre-set as a reference value for and/or in the software. Preferably, in the aforesaid manner, the intensities of the control and test lines may be measured and determined, and the diagnostic results (such as positive, negative, invalid) can be determined and/or obtained.

Preferably, two or more images may be taken for each lateral flow cassette which is detected and/or determined to be of the fluorescent and/or emission signal type. The first image is preferably an image of the cassette with the white LED on, the color and/or UV LEDs off, and the optical filter away from the camera. This image is preferably used for automatic cassette recognition, which may preferably be based on information such as cassette size, shape, writings, and other specific features. Preferably, with the color and/or UV LEDs off, this image will not show any line in the membrane area for fluorescent lateral flow cassettes.

Preferably, the second image is taken with the white LEDs off, the color and/or UV LEDs on, and the optical filter in front of the camera. The color and/or UV LEDs preferably illuminate only the membrane area of the cassette, preferably to reduce an auto-fluorescence background signal such as from a plastic case of the cassette. Intensities of the fluorescent control line and/or test lines are preferably obtained in the second image.

The image processing software preferably combines the two images, and presents a user with the cassette type and/or the diagnostic results.

In doing so, the image processing software preferably accesses, references and/or consults one or more databases which are (a) local to the image processing software, to the device, to other components of the system, and/or to the computer readable medium, and/or (b) remote therefrom, with a communications element being used to remotely access, reference and/or consult them. The databases preferably take the form of one or more local, remote, distributed, congruent and/or peer-to-peer databases which are preferably be accessible by the image processing software and/or the device locally and/or over one or more of its regular wireless (and/or wired) communication networks, including terrestrial and/or satellite networks—e.g., the Internet and cloud-based networks.

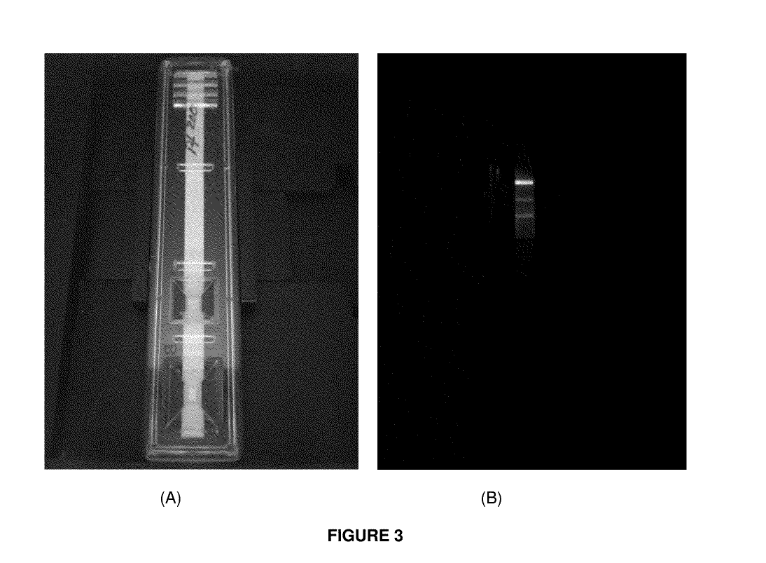

FIG. 3 shows an example of two images such as may be taken of one type of fluorescent lateral flow cassette. FIG. 3(A) shows an image of the cassette. Preferably, the FIG. 3(A) image is taken with white LEDs on, color LEDs off, and optical filter away from the camera. The image in FIG. 3(A) could preferably be used for cassette recognition. FIG. 3(B) shows a fluorescent signal image of the same cassette. The FIG. 3(B) image is preferably taken with white LEDs off, color LEDs on, and optical filter in front of the camera. Preferably, in the shown fluorescent lateral flow test, Europium dye is used. The image in FIG. 3(B) could preferably be used for diagnostic test result analysis.

Functional Check And Fluorescent Signal Calibration

A functional check and calibration (“FCC”) cassette is preferably used to make sure the device, system and/or method are functional for both gold (reflection signal) and fluorescent (emission signal) lateral flow cassette test types. The FCC cassette preferably contains at least one color line and one fluorescent signal line. The plastic case of the FCC cassette may or may not be the same as that of a normal diagnostic cassette, as long as an associated software (“SW”) algorithm [which may be provided according to one preferred embodiment of the invention] is preferably able to differentiate it therefrom. Preferably, the intensities of the color line and fluorescent line are pre-set, and should not appreciably change over a meaningful time relative to the expected lifetime(s) of the FCC cassette and/or the device according to the present invention. Preferably, to evaluate the device, the intensities of the two lines are tested. The criteria for functional check pass and/or failure is preferably pre-determined, and recorded in and/or accessed by the software.

### Other Preferred Embodiments

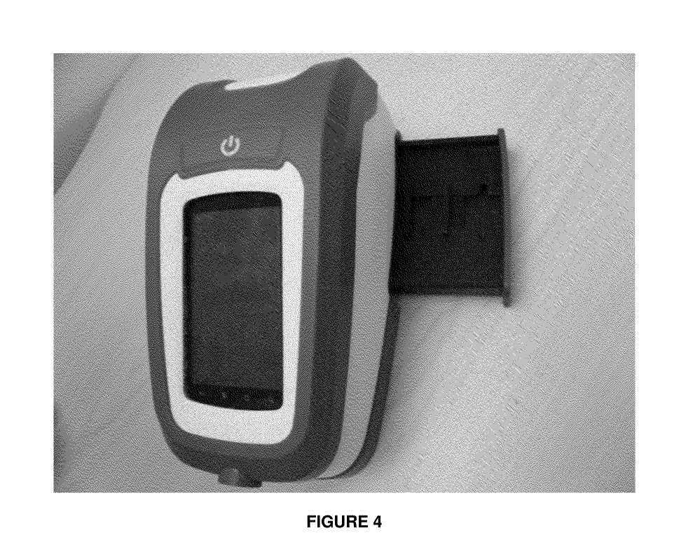

A lateral flow reader device, system and/or method according to one preferred embodiment of the invention may comprise, be used in conjunction with, and/or be based on a cell phone. FIG. 4 shows this preferred embodiment. The cell phone's back camera may be used as the color camera according to this embodiment. Preferably, the cell phone's multiple functions (such as image taking, image processing, and/or information transferring) may afford benefits for the device, system and/or method according to this embodiment.

In this preferred embodiment, the cassette drawer and the enclosed cassette chamber preferably provide a constant lighting condition, with LEDs on, only when an image is being taken. Preferably, the device, system and/or method may be adapted such that the left channel audio signal from the cell phone may be used to control whether the white LEDs are on or off, and/or such that the right channel audio signal from the cell phone may be used to control whether the color and/or UV LEDs are on or off.

A device, system and/or method according to another preferred embodiment of the invention may be custom-designed and/or assembled by using separate parts and/or components, so as to afford the same, similar, or greater functionality than those comprising, used in conjunction with, and/or based on commercially available cell phones.

A further preferred embodiment of the device, system and/or method according to the invention may comprise, be used in conjunction with, and/or be based on a scanner device. In this embodiment, a scanning head may preferably be equipped with a red (R) LED, a green (G) LED, a blue (B) LED, and a UV LED. Preferably, for color images of gold (or reflection signal) lateral flow cassettes, only the RGB LEDs (or white LED) may be on when scanning Preferably, for fluorescent (or emission signal) lateral flow cassette detection, two or more scans may be taken. Preferably, for cassette recognition, the first scan obtains a color image of the cassette, including its plastic case, with the RGB LEDs (or white LED) on and the UV LED off. The second scan preferably obtains the emitted fluorescent signal of the control line and the test line in the membrane area of the cassette, with the RGB LEDs (or white LED) off and the UV LED on. In this preferred embodiment, a band pass filter is placed in from of the image sensor when the UV LED is on.

Yet another preferred embodiment may be provided with a filter wheel which has two or more, and preferably several, optical band pass filters for different fluorophores and a through-hole (or clear) position for bright and/or white-light images. This embodiment may preferably also contain two or more, and preferably several, color and/or UV LEDs for different fluorophores. Whether each of these LEDs is on, or off, may preferably be controlled by the device software of this embodiment. Preferably, the filter wheel switch may be motorized, or the filter wheel may be turned manually. The filter wheel may preferably be turned automatically to the right filter depending on which LED(s) are on and/or which cassette is recognized.

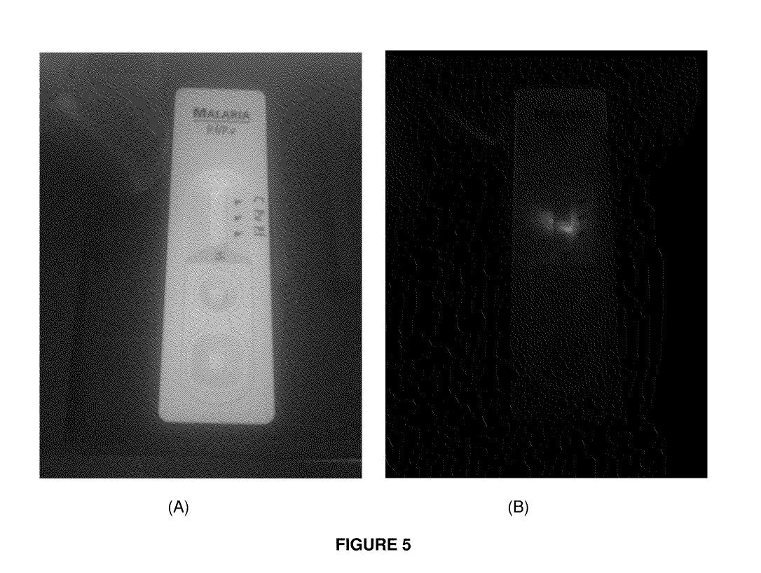

A yet further preferred embodiment of the invention may be provided without any optical filter sliding switch, without any optical filter wheel, and without any band pass filters. Instead, the device, system and/or method may comprise, be used in conjunction with, and/or be based on a white LED, a UV LED, and a long pass filter. Preferably, in this embodiment, the optical filter may be always placed in front of the camera. As an example, the wavelength of the UV LED may be about 375 nanometers (nm), and the cutting edge wavelength of the optical long pass filter may be about 420 nanometers (nm). In this embodiment of the invention, the fluorophore of the fluorescent lateral flow test preferably may be Europium dye. FIG. 5 shows preferable images of a fluorescent lateral flow cassette according to this embodiment. FIG. 5(A) shows a preferable image of the cassette when the white LED is on, the UV LED is off, and the 420 nanometer (nm) long pass filter is placed in front of camera. FIG. 5(B) shows a preferable image of the cassette when the white LED is off, the UV LED is on, and the 420 nanometer (nm) long pass filter is placed in front of the camera. Preferably, the image in FIG. 5(A) may be used for cassette recognition, and/or the image in FIG. 5(B) may be used for fluorescent signal detection, according to this embodiment of the invention.

In still another preferred embodiment, the color camera may have a zoom-in function and/or be able to take a zoomed-in image of the membrane area when the color and/or UV LEDs are on, preferably for better fluorescence detection performance.

In a still further preferred embodiment, there may be provided one or more optical fibers and/or an optical fiber bundle to guide light to a specific area.

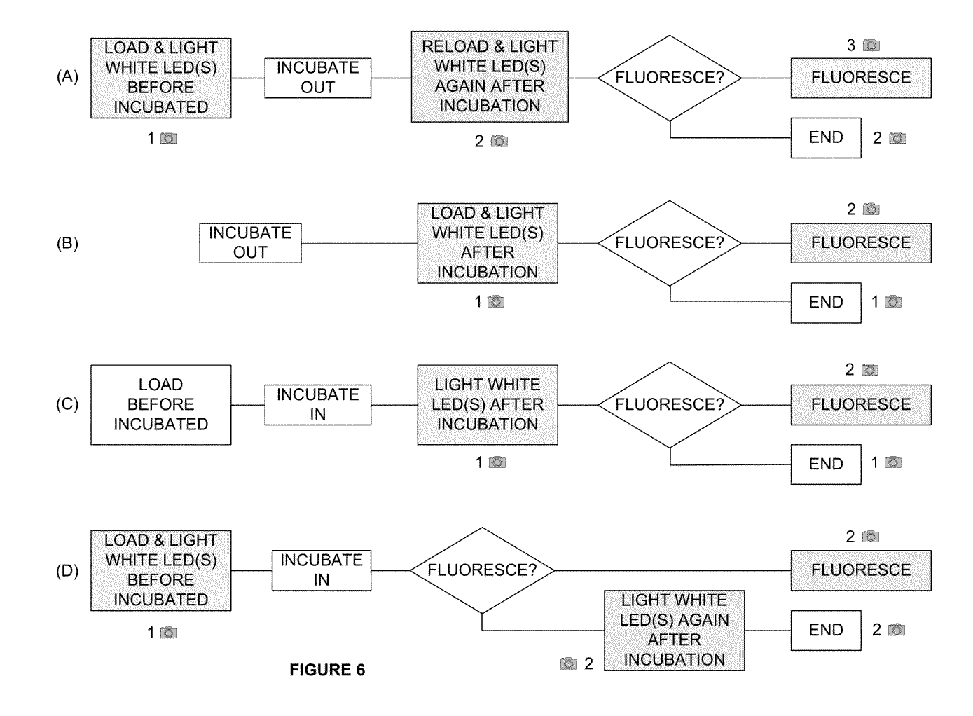

FIG. 6 shows four potential workflows for use in or in association with the device, system and/or method according to the invention. FIGS. 6(A) and 6(B) show workflows where the cassette is preferably incubated outside of a reader device according to the invention. In FIG. 6(A), the cassette is loaded into the device and the white LEDs are illuminated before the cassette is fully incubated. An image taken at this stage may be used for cassette recognition. In FIG. 6(A), the cassette is removed from the device and reloaded again after incubation. In FIG. 6(B), the cassette is first loaded into the device after incubation. In both cases, after incubation, the white LEDs are illuminated and images taken for cassette recognition.

FIGS. 6(C) and 6(D) show workflows where the cassette is preferably incubated inside of a reader device according to the invention. In both cases, the cassette is loaded into the device before it is fully incubated. Images taken when the white LEDs are first illuminated may be used for cassette recognition. In FIG. 6(D) the white LEDs are first illuminated before the cassette is fully incubated, whereas in FIG. 6(C) the white LEDs are first illuminated after incubation. In FIG. 6(D), for reflection signal tests, the white LEDs are illuminated again after incubation.

In FIGS. 6(A) through 6(D), for reflection signal tests, images taken when the white LEDs are illuminated after incubation may be analyzed by the image processing software. For emission signal tests, the color and/or UV LEDs are illuminated to fluoresce the test and control lines and, with the optical filter in front of the camera, an image is taken for analysis by the image processing software.

In the workflow shown in FIG. 6(A), two images are taken for reflection signal tests and three for emission signal tests. In the workflows shown in FIGS. 6(B) and 6(C), one image is taken for reflection signal tests and two for emission signal tests. In the workflow shown in FIG. 6(D), two images are taken for reflection signal tests and two for emission signal tests.

Computer Readable Medium

The computer readable medium (e.g., CD-ROM, DVD-ROM, flash USB stick, RAM, ROM, and/or other computer memory device) includes executable instructions which are physically stored thereon and which, upon execution, preferably encode processors to perform the method according to the invention.

All of the aforementioned, depicted and various structures, configurations, relationships, processes, utilities and the like may be, but are not necessarily, incorporated into and/or achieved by the invention. Any one or more of the aforementioned structures, configurations, relationships, processes, utilities and the like may be implemented in and/or by the invention, on their own, and/or without reference, regard or likewise implementation of any of the other aforementioned structures, configurations, relationships, processes, utilities and the like, in various permutations and combinations, as will be readily apparent to those skilled in the art, without departing from the pith, marrow, and spirit of the disclosed invention.

This concludes the description of presently preferred embodiments of the invention. The foregoing description has been presented for the purpose of illustration and is not intended to be exhaustive or to limit the invention to the precise form disclosed. Other modifications, variations and alterations are possible in light of the above teaching and will be apparent to those skilled in the art, and may be used in the design and manufacture of other embodiments according to the present invention without departing from the spirit and scope of the invention. It is intended the scope of the invention be limited not by this description but only by any claims forming a part of this application, and/or the claims of any application claiming priority from this application, and/or any patent issuing thereon.

1. A device for analysis of various test cassettes for immunoassay rapid diagnostic tests, the device comprising:
one or more walls defining an enclosed chamber which is adapted to selectively receive the various cassettes one at a time, each as a loaded cassette, in selectively removable relation;
two or more light emitting diodes (LEDs) within the chamber, which comprise at least one reflection light emitting diode (LED) adapted to illuminate the loaded cassette when lit, and at least one fluorescent LED adapted to fluoresce the loaded cassette when lit;
an imaging element inside the chamber which automatically captures a first image of the loaded cassette when at least one of the LEDs is lit; and
at least one processor which, with reference to the first image and to a database that comprises at least one test protocol associated with each of the various test cassettes, automatically identifies an applicable one said test protocol for the loaded cassette;
wherein the processor automatically, after incubation of the loaded cassette and depending on the applicable one said test protocol:
(i) determines when the first image captures a post-incubation test signal from the loaded cartridge; and otherwise
(ii) lights the fluorescent LED to generate the test signal by emission, or the reflection LED to generate the test signal by reflection, from the loaded cassette and uses the imaging element to capture a second image of the loaded cassette and of the test signal; and
wherein the processor automatically provides the test signal, in the first image or the second image, for analysis of the loaded cassette.

2. A device according to claim 1, wherein the processor automatically analyzes the test signal with reference to the applicable one said test protocol for the loaded cassette.

3. A device according to claim 2, wherein the test signal comprises a test line signal corresponding to a test line present on the loaded cassette after incubation, and the processor automatically measures an intensity of the test line signal.

4. A device according to claim 2, wherein the test signal comprises a control line signal corresponding to a control line present on the loaded cassette after incubation, and the processor automatically measures an intensity of the control line signal.

5. A device according to claim 2, wherein the applicable one said test protocol comprises a predetermined assay threshold value, and wherein the processor analyzes the test signal with reference to the assay threshold value to automatically determine a diagnostic test result associated with the loaded cassette.

6. A device according to claim 5, further comprising an output device which automatically presents the diagnostic test result to a user of the device.

7. A device according to claim 2, further comprising a memory onboard the device which stores one or more sets of executable instructions to encode the processor to automatically analyze the test signal.

8. A device according to claim 1, further comprising a memory onboard the device which stores one or more sets of executable instructions to encode the processor to automatically identify the applicable one said test protocol.

9. A device according to claim 1, further comprising a memory onboard the device which stores the database.

10. A device according to claim 1, further comprising a communications element onboard the device which the processor automatically uses to remotely reference the database.

11. A device according to claim 1, further comprising at least one optical filter, and wherein (i) the processor automatically ensures the optical filter is moved to an engaged position between the loaded cassette and the imaging element, before lighting a corresponding one said emission LED, and the imaging element then captures the test signal through the optical filter, and (ii) the processor automatically ensures the optical filter is moved to a disengaged position clear of the imaging element, before lighting the reflection LED, and the imaging element then captures the test signal clear of the optical filter.

12. A device according to claim 11, wherein the optical filter is mounted on a sliding switch which slides the optical filter between the engaged position and the disengaged position.

13. A device according to claim 11, wherein the optical filter is mounted on a rotatable mechanism which rotates the optical filter between the engaged position and the disengaged position.

14. A device according to claim 11, wherein the optical filter is an optical band pass filter.

15. A device according to claim 1, further comprising an optical long pass filter positioned between the loaded cassette and the imaging element, and wherein the imaging element captures the test signal through the optical long pass filter.

16. A device according to claim 1, wherein said at least one reflection LED comprises at least one white LED.

17. A device according to claim 1, wherein said at least one fluorescent LED comprises at least one ultraviolet LED.

18. A device according to claim 1, wherein said at least one fluorescent LED comprises at least one colored LED.

19. A device according to claim 1, wherein the processor identifies the applicable one said test protocol with reference to one or more of the following which are captured in the first image, and stored in the database, for the loaded cassette: one or more cassette dimensions, one or more cassette shapes, one or more detection line dimensions, one or more control line dimensions, one or more detection areas, one or more membrane areas, one or more control line positions, one or more test line positions, one or more cassette colors, one or more line colors, manufacturer indicia, product indicia, brand name indicia, application indicia, disease indicia, test type indicia, incubation time indicia, expected results indicia, barcodes, two-dimensional barcodes, labels, and other printed and written indicia.

20. A device according to claim 1, wherein the processor additionally identifies the applicable one said test protocol with reference to one or more of the following which are received from the loaded cassette, and stored in the database for the loaded cassette: magnetically stored data, fluorescence data, and radioactive signal data.

21. A device according to claim 1, adapted for analysis of various lateral flow cassettes as the test cassettes.

22. A device according to claim 1, adapted for use with a cellular telephone to provide at least one of said imaging element and said processor.

23. A device according to claim 22, wherein left and right channel audio signals from the cellular telephone are adapted to turn the reflection LED and the fluorescent LED on and off.

24. A device according to claim 1, wherein the imaging element comprises one or more scanning heads.

25. A system for analysis of various test cassettes for immunoassay rapid diagnostic tests, the system comprising:
one or more walls defining an enclosed chamber which is adapted to selectively receive the various cassettes one at a time, each as a loaded cassette, in selectively removable relation;
two or more light emitting diodes (LEDs) within the chamber, which comprise at least one reflection light emitting diode (LED) adapted to illuminate the loaded cassette when lit, and at least one fluorescent LED adapted to fluoresce the loaded cassette when lit;
a database that comprises at least one test protocol associated with each of the various test cassettes;
a cellular telephone received by the walls of the chamber, with the cellular telephone comprising: a camera inside the chamber which automatically captures a first image of the loaded cassette when at least one of the LEDs is lit; and at least one processor which, with reference to the first image and in communication with the database, automatically identifies an applicable one said test protocol for the loaded cassette;
wherein the processor automatically, after incubation of the loaded cassette and depending on the applicable one said test protocol:
(i) determines when the first image captures a post-incubation test signal from the loaded cartridge; and otherwise
(ii) lights the fluorescent LED to generate the test signal by emission, or the reflection LED to generate the test signal by reflection, from the loaded cassette and uses the imaging element to capture a second image of the loaded cassette and of the test signal; and
wherein the processor automatically provides the test signal, in the first image or the second image, for analysis of the loaded cassette.

26. A system according to claim 25, wherein left and right channel audio signals from the cellular telephone are adapted to turn the reflection LED and the fluorescent LED on and off.

27. A method for analysis of various test cassettes for immunoassay rapid diagnostic tests, the method comprising:
a receiving step of selectively receiving the various cassettes one at a time, each as a loaded cassette, in selectively removable relation within an enclosed chamber which is defined by one or more walls;
a light emitting diode (LED) step of providing two or more LEDs within the chamber, which comprise at least one reflection LED adapted to illuminate the loaded cassette when lit, and at least one fluorescent LED adapted to fluoresce the loaded cassette when lit;
an imaging step of automatically capturing a first image of the loaded cassette, when at least one of the LEDs is lit, using an imaging element inside the chamber;
a database step of providing a database which comprises at least one test protocol associated with each of the various test cassettes; and
a processing step of using at least one processor, with reference to the first image and to the database, automatically identifying an applicable one said test protocol for the loaded cassette;
wherein in the processing step, the processor automatically, after incubation of the loaded cassette and depending on the applicable one said test protocol:
(i) determines when the first image captures a post-incubation test signal from the loaded cartridge; and otherwise
(ii) lights the fluorescent LED to generate the test signal by emission, or the reflection LED to generate the test signal by reflection, from the loaded cassette and uses the imaging element to capture a second image of the loaded cassette and of the test signal; and
wherein in the processing step, the processor automatically provides the test signal, in the first image or the second image, for analysis of the loaded cassette.

28. A method according to claim 27 wherein, in the processing step, the processor automatically analyzes the test signal with reference to the applicable one said test protocol for the loaded cassette.

29. A method according to claim 28 wherein, in the processing step, the test signal comprises a test line signal corresponding to a test line present on the loaded cassette after incubation, and the processor automatically measures an intensity of the test line signal.

30. A method according to claim 28 wherein, in the processing step, the test signal comprises a control line signal corresponding to a control line present on the loaded cassette after incubation, and the processor automatically measures an intensity of the control line signal.

31. A method according to claim 28 wherein, in the processing step, the applicable one said test protocol comprises a predetermined assay threshold value, and the processor analyzes the test signal with reference to the assay threshold value to automatically determine a diagnostic test result associated with the loaded cassette.

32. A method according to claim 31, further comprising a presentation step, wherein the diagnostic test result is automatically presented using an output device.

33. A method according to claim 28 wherein, before the processing step, one or more sets of executable instructions are stored in a memory and, in the processing step, the executable instructions encode the processor to automatically analyze the test signal as aforesaid.

34. A method according to claim 27 wherein, before the processing step, one or more sets of executable instructions are stored in a memory and, in the processing step, the executable instructions encode the processor to automatically identify the applicable one said test protocol.

35. A method according to claim 27 wherein, in the processing step, the processor automatically uses a communications element to remotely reference the database.

36. A method according to claim 27, further comprising a filtering step of providing at least one optical filter, and wherein in the processing step, (i) the processor automatically ensures the optical filter is moved to an engaged position between the loaded cassette and the imaging element, before lighting a corresponding one said emission LED, and the imaging element then captures the test signal through the optical filter, and (ii) the processor automatically ensures the optical filter is moved to a disengaged position clear of the imaging element, before lighting the reflection LED, and the imaging element then captures the test signal clear of the optical filter.

37. A method according to claim 27, further comprising a filtering step of providing an optical long pass filter positioned between the loaded cassette and the imaging element, and wherein in the processing step, the imaging element captures the test signal through the optical long pass filter.

38. A method according to claim 27 wherein, in the LED step, at least one white LED is provided as said at least one reflection LED.

39. A method according to claim 27 wherein, in the LED step, at least one ultraviolet LED is provided as said at least one fluorescent LED.

40. A method according to claim 27 wherein, in the LED step, at least one colored LED is provided as said at least one fluorescent LED.

41. A method according to claim 27 wherein, in the processing step, the processor identifies the applicable one said test protocol with reference to one or more of the following which are captured in the first image, and stored in the database, for the loaded cassette: one or more cassette dimensions, one or more cassette shapes, one or more detection line dimensions, one or more control line dimensions, one or more detection areas, one or more membrane areas, one or more control line positions, one or more test line positions, one or more cassette colors, one or more line colors, manufacturer indicia, product indicia, brand name indicia, application indicia, disease indicia, test type indicia, incubation time indicia, expected results indicia, barcodes, two-dimensional barcodes, labels, and other printed and written indicia.

42. A method according to claim 27 wherein, in the processing step, the processor additionally identifies the applicable one said test protocol with reference to one or more of the following which are received from the loaded cassette, and stored in the database for the loaded cassette: magnetically stored data, fluorescence data, and radioactive signal data.

43. A method according to claim 27 wherein, in at least one of the imaging step and the processing step, a cellular telephone is provided as said imaging element and/or as said processor.

44. A method according to claim 43 wherein, in at least one of the imaging step and the processing step, left and right channel audio signals from the cellular telephone turn the reflection LED and the fluorescent LED on and off.

45. A method according to claim 27 wherein, in the imaging step, one or more scanning heads are provided as at least part of said imaging element.

46. A computer readable medium for analysis of various test cassettes for immunoassay rapid diagnostic tests, and for use with: one or more walls defining an enclosed chamber which is adapted to selectively receive the various cassettes one at a time, each as a loaded cassette, in selectively removable relation; two or more light emitting diodes (LEDs) within the chamber, which comprise at least one reflection light emitting diode (LED) adapted to illuminate the loaded cassette when lit, and at least one fluorescent LED adapted to fluoresce the loaded cassette when lit; an imaging element inside the chamber; and a database that comprises at least one test protocol associated with each of the various test cassettes;
with the computer readable medium comprising executable instructions which are physically stored thereon and which, upon execution, encode at least one processor to automatically:
capture a first image of the loaded cassette using the imaging element when at least one of the LEDs is lit;
identify, with reference to the first image and to the database, an applicable one said test protocol for the loaded cassette;
after incubation of the loaded cassette and depending on the applicable one said test protocol: (i) determine when the first image captures a post-incubation test signal from the loaded cartridge; and otherwise (ii) light the fluorescent LED to generate the test signal by emission, or the reflection LED to generate the test signal by reflection, from the loaded cassette and uses the imaging element to capture a second image of the loaded cassette and of the test signal; and
provide the test signal, in the first image or the second image, for analysis of the loaded cassette.
